SEQUENCE LISTING (8 sequences)

SEQ ID NO: 1 - protein, 152 aa, Homo sapiens [sequence omitted]

SEQ ID NO: 2 - protein, 20 aa, Homo sapiens
MYRMQLLSCIALSLALVTNS

SEQ ID NO: 3 - DNA, 459 bp, Homo sapiens
gccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctgtga

SEQ ID NO: 4 - DNA, 60 bp, Homo sapiens
atgtacaggatgcaactcctgtcttgcattgcactaagtcttgcacttgtcacaaacagt

SEQ ID NO: 5 - DNA, 59 bp, Artificial Sequence
  Artificial primer
ctttggcttactcaaactcagaatgaaacaaatcgaagacaagatcgaagaaatccttt

SEQ ID NO: 6 - DNA, 59 bp, Artificial Sequence
  Artificial primer
aaaggatttcttcgatcttgtcttcgatttgtttcattctgagtttgagtaagccaaag

SEQ ID NO: 7 - DNA, 86 bp, Artificial Sequence
  Artificial primer
ctactagcatgtacaggatgcaactcctgtcttgcattgcactaagtcttgcacttgtcacaaacagtgccgttcagggtccagaa

SEQ ID NO: 8 - DNA, 54 bp, Artificial Sequence
  Artificial primer
catgccatgctcacttgtcatcgtcatccttgtaatccagcagtttcaatgcac

(19) United States
(12) Patent Application Publication
(10) Pub. No.: US 2020/0362014 A1
(43) Pub. Date: Nov. 19, 2020

(54) APPLICATIONS OF SOLUBLE PROTEIN BAFF IN B CELL IN-VITRO CULTURE AND PROLIFERATION

(71) Applicant: Shenzhen City of Regeneration Biological Pharmacuetical Technology Co., Ltd., Shenzhen (CN)

(72) Inventors: Hui ZHANG, Guangzhou City (CN); Yiwen ZHANG, Guangzhou City (CN); Shilin GAO, Guangzhou City (CN)

(21) Appl. No.: 16/476,853

(22) PCT Filed: Nov. 7, 2016
(86) PCT No.: PCT/CN2016/104807

§ 371 Date: Jul. 9, 2019

(30) Foreign Application Priority Data
Aug. 18, 2016 (CN) 201610692755.1

(51) Int. Cl. C07K 14/705; C07K 14/55; C12N 15/85; C12N 5/0781

(52) U.S. Cl. CPC C07K 14/70575; C07K 14/55; C12N 15/85; C12N 5/0635; C12N 2501/33; C12N 2500/24; C12N 2501/2302; C12N 2501/2304; C07K 2319/03

(57) ABSTRACT

Disclosed in the present invention is an application of soluble protein BAFF in B cell in-vitro culture and proliferation. Peptide expressed by secretory BAFF peptide in the present invention can be effectively secreted out of cells to generate corresponding bioactivity. During the process of culture, a cell line in the present invention can continuously secrete water-soluble BAFF protein having bioactivity, the water-soluble BAFF protein served as tool cells are co-cultured with B cells, and in-vitro proliferation and antigen presentation effects of human B cells can be effectively improved.

### FIELD OF THE INVENTION

The present invention relates to a construction method of a cell and application thereof, and in particular to a construction method of a secretory BAFF cell line and its application in improving in-vitro proliferation and antigen presentation of human peripheral blood B cells.

### BACKGROUND OF THE INVENTION

In recent years, more and more research groups have tried to use adoptive T-cell immunotherapy to cure tumor or virus infection since T cells play an important role in the immune system. At present, multiple domestic and international research groups are researching how to maintain long-term in-vitro culture of T cells, extend their survival time, and improve their immunological function for the purpose of T-cells' application in adoptive immunotherapy. In T-cell therapy, the main strategy is to acquire enough T cells having killing activity through clonal proliferation and then co-culture them with antigen presenting cells. Massive proliferation of endogenous antigen presenting cells in human body for T-cell immunotherapy relative to constructing artificial antigen presenting cells has the advantages of the least introduction of exogenous cells, minimum interference by alloantigen, a presenting process being closer to physiological status, and a higher success rate of proliferation. Among three types of main professional antigen presenting cells in human body, B cells exist in large numbers, are easily acquired, and hold excellent proliferation potential, so it will not take great efforts to get numerous B cells with an antigen presentation function. It has been proved that B cells play important roles in presenting the antigens to T cells, improving in-vitro culture time and proliferation efficiency of T cell, boosting T-cell functions. Proliferating human peripheral blood B cells having antigen specificity in vitro massively, and improving antigen presentation function thereof is crucial to the T cell adoptive therapy of tumor and infection.

Therefore, how to massively proliferate human peripheral blood B cells with the antigen specificity, and improve the antigen presentation function thereof becomes a hot research topic in recent years. In 1997, Shultze firstly reported a method for massively proliferating in-vitro B cells by stimulating and activating a NIH-3T3 cell capable of stably expressing CD40L, and proved that in-vitro cultured B cells could highly express CD80, CD86 and other essential molecules for antigen presenting (Schultze et al., 1997). Later, von Bergwelt-Baildon and other researchers made several improvements to the system. Although the in-vitro proliferation efficiency of B cells is greatly increased after the improvement, B cell apoptosis caused by activation has to be reduced for the purpose of further increasing the proliferation efficiency of B cells due to highly expressed Fas by activated B cells, and the most likely apoptosis of B cells, the impact of which however may not be offset by continuous stimulation of CD40L (Garrone et al., 1995; Zhang et al., 1996).

The B-cell activating factor (BAFF) belongs to TNF superfamily, and has three receptors which are TACI, BCMA and BAFF-R respectively. Wherein, BAFF bonded to BAFF-R is a primary cause of promoting B cell survival. In addition to promoting B cell survival, BAFF may also cooperate with CD40L to increase the proliferation efficiency of B cells (Do et al., 2000; Moore et al., 1999). BAFF is a transmembrane protein, and can release a water-soluble fragment with activity under the action of proteolytic enzyme. Because of such a special feature, BAFF has never been introduced in the system of human B cell in-vitro proliferation and long-term culture yet.

### SUMMARY OF THE INVENTION

The purpose of the present invention is to provide an application of a soluble protein BAFF in B cell in-vitro culture and proliferation.

A technical solution used by the present invention is as follows:

A secretory BAFF peptide, wherein the secretory BAFF peptide comprises an amino acid sequence consisting of amino acids 133-285 of BAFF and an IL-2 peptide at N-terminal thereof. A sequence of the amino acids 133-285 of BAFF is AVQGPEETVTQDCLQLIADSETPTIQKGSYTFVPWLLSFKRGSALEEKENKILVKETGYFFIY GQVLYTDKTYAMGHLIQRKKVHVFGDELSLVTLFRCIQNMPETLPNNSCYSAGIAKLEEGD ELQLAIPRENAQISLDGDVTFFGALKLL (SEQ ID NO: 1), and an amino acid sequence of the IL-2 peptide is MYRMQLLSCIALSLALVTNS (SEQIDNO: 2).

An expression vector for a secretory BAFF peptide, wherein the expression vector comprises a nucleotide sequence expressing the above secretory BAFF peptide.

As further improvement of the above expression vector, wherein the expression vector further comprises a nucleotide sequence expressing a membrane type CD40L.

A cell for expressing a secretory BAFF peptide, wherein the cell is transfected with the above expression vector.

As further improvement of the above cell, the cell is further transfected with a vector expressing a membrane type CD40L.

As further improvement of the above cell, the cell is 293T cell.

A method for proliferating human peripheral blood B cells in vitro, wherein the method comprises a step of co-culturing the human peripheral blood B cells with cells simultaneously expressing a secretory BAFF peptide and a membrane type CD40L.

As further improvement of the above method for proliferating human peripheral blood B cells in vitro, human peripheral blood B cells are cultured in insulin-transferrin-supporting IMDM medium supplemented with CpG2006/2219 and ciclosporin A for stimulating B cells proliferation, and with human IL-4, IL-2 and IL-10 for maintaining cell growth.

The beneficial effects of the present invention are as follows:

Peptide expressed by secretory BAFF peptide in the present invention can be effectively secreted out of cells to generate corresponding bioactivity

During the process of culture, the cell line in the present invention can continuously secrete water-soluble BAFF protein having bioactivity, the water-soluble BAFF protein served as tool cells are co-cultured with B cells, and in-vitro proliferation and antigen presentation effects of human B cells can be effectively improved.

### BRIEF DESCRIPTION OF THE DRAWINGS

FIG. 1 is a schematic diagram of CD40L and soluble BAFF expression plasmid.

FIG. 2 shows the proliferation for co-culturing B cells with cell lines 293T, 293T-CD40L or 293T-CD40L-sBAFF used as feed layer cells; A) the proliferation curve of B cells; and B) B cell proliferation folds calculated after co-culturing for 35 days.

FIG. 3 shows flow CFSE detection results of proliferation for co-culturing B cells with cell lines 293T, 293T-CD40L or 293T-CD40L-sBAFF used as the feed layer cells for 7 days, A) Percentage of proliferated B cells analyzed with flow cytometry, and B) Statistical results of percentage.

FIG. 4 shows expression of CD80, CD86, CD70 and CD275 detected by flow cytometry on the 5th day or the 30th day, after B cells are co-cultured with cell lines 293T, 293T-CD40L or 293T-CD40L-sBAFF used as feed layer cells.

FIG. 5 shows that B cells cultured by the cell line 293T-CD40L-sBAFF are used as the antigen presenting cells to cultivate CD8 cells; a, b) IFN-γ secretion of CTL cultivated by B cells in different culture conditions; c) CTL killing ability detection; d) Schematic diagram of a CTL in-vivo model; and e) tumor volume detection in in-vivo experiment.

### DETAILED DESCRIPTION OF THE EMBODIMENTS

A secretory BAFF peptide, wherein the secretory BAFF peptide comprises an amino acid sequence consisting of amino acids 133-285 of BAFF and an IL-2 peptide coupled at the N-terminal thereof. A sequence of the amino acids 133-285 of BAFF is AVQGPEETVTQDCLQLIADSETPTIQKGSYTFVPWLLSFKRGSALEEKENKILVKETGYFFIY GQVLYTDKTYAMGHLIQRKKVHVFGDELSLVTLFRCIQNMPETLPNNSCYSAGIAKLEEGD ELQLAIPRENAQISLDGDVTFFGALKLL, and an amino acid sequence of the IL-2 peptide is MYRMQLLSCIALSLALVTNS.

As an example, the nucleotide sequence of the amino acids 133-285 of BAFF isgccgttcagggtccagaagaaacagtcactcaagactgcttgcaactgattgcagacagtgaaacaccaactatacaaaaaggatcttacaca tt tgttccatggcttctcagctaaaaggggaagtgccctagaagaaaaagagaataaaatattggtcaaagaaactggttacttttttatatatggtca ggttttatatactgataagacctacgccatgggacatctaattcagaggaagaaggtccatgtctttggggatgaattgagtctggtgactttgtttcg atgtattcaaaatatgcctgcctgctattcagctggcattgcaaaactggaagaaggagatgaactccaacttgcaata ccaagagaaaatgcacaaatatcactggatggagatgtcacattttttggtgcattgaaactgctgtga (SEQ ID NO: 3). The nucleotide sequence of the IL-2 peptide is atgtacaggatgcaactcctgtcttgcattgcactaagtcttgcacttgtcacaaacagt (SEQ IDNO: 4).

An expression vector for a secretory BAFF peptide, wherein the expression vector comprises a nucleotide sequence expressing the above secretory BAFF peptide.

As further improvement of the above expression vector, wherein the expression vector further comprises a nucleotide sequence expressing a membrane type CD40L.

A cell for expressing a secretory BAFF peptide, wherein the cell is transfected with the above expression vector.

As further improvement of the above cell, the cell is further transfected with a vector expressing a membrane type CD40L.

As further improvement of the above cell, the cell is 293T cell.

On the basis of the existing method for proliferating human peripheral blood B cells in vitro, human peripheral blood B cells are co-cultured with cells simultaneously expressing a secretory BAFF peptide and a membrane type CD40L, and the in vitro proliferation and antigen presentation effect of human B cells can be effectively improved.

As further improvement of the above method for proliferating human peripheral blood B cells in vitro, human peripheral blood B cells are cultured in insulin-transferrin-supporting IMDM medium supplemented with CpG2006/2219 and ciclosporin A for stimulating B cells proliferation, and with human IL-4, IL-2 and IL-10 for maintaining cell growth.

The technical solution of the present invention is further described below in combination with experiments.

### Construction of Vector

Templates of molecular cloning experiments related to the present invention were cDNA of PBMC cells. The PCR enzyme involved was 2× Premix PrimeSTAR® HS. Primers involved were shown in the following table.

Sequence table of primers synthetized
by the experiment

| | | SEQ |
|---|---|---|
| | | ID |
| Name | Sequence | NO: |
| CD40L- | CTTTGGCTTACTCAAACTCAGAATGAAACA | 5 |
| F-NheI | AATCGAAGACAAGATCGAAGAAATCCTTT | |
| CD40L- | AAAGGATTTCTTCGATCTTGTCTTCGATTTGT | 6 |
| R-AgeI | TTCATTCTGAGTTTGAGTAAGCCAAAGCTAC | |
| | TAGCATGTACAGGATGCAACTCCTGTCTTGC | |
| BAFF-F- | ATTGCACTAAGTCTTGCACTTGTCACAAACA | 7 |
| ss-NheI | GTGCCGTTCAGGGTCCAGAA | |
| BAFF-R- | CATGCCATGCTCACTTGTCATCGTCATCCTT | 8 |
| Flag-Agel | GTAATCCAGCAGTTTCAATGCAC | |

The PCR system is shown in the following table:

Membrane type CD40LPCR system

| Reagent | Dosage |
|---|---|
| cDNA template | 200 ng |
| CD40L-F-NheI | 1 μl |
| CD40L-R-AgeI | 1 μl |
| Premix PrimeSTAR ® HS | 25 μl |
| H₂O | Dilute to 50 μl |

The PCR conditions were 94° C. 30 s, 62° C. 30 s, 72° C. 1 min, 72° C. 7 min, and finally allowed to stand at 4° C.

Secretory BAFF PCR system

| Reagent | Dosage |
|---|---|
| cDNA template | 200 ng |
| BAFF-F-ss-Nhel | 1 μl |
| BAFF-R-Flag-Agel | 1 μl |
| Premix PrimeSTAR ® HS | 25 μl |
| H₂O | Dilute to 50 μl |

The PCR conditions were 94° C. 30 s, 58° C. 30 s, 72° C. 30 s, 72° C. 7 min, and finally allowed to stand at 4° C.

Upon the end of PCR, electrophoresis was performed on PCR products to identify and cut the target gene band under blue light. A gel extraction kit was used for extraction.

After the target fragment was obtained by gel extraction, a double enzyme digestion reaction was respectively performed on the target gene fragment and the corresponding vector simultaneously.

After extracting enzyme digestion products, the obtained target gene fragment was ligated with the obtained target vector fragment overnight at 16° C. by using T4 DNA ligase of Takara. Wherein, the CD40L fragment was connected with the cPPT-mini-MMIG vector, and the BAFF fragment was connected with the cPPT-IRES-RFP vector.

### Construction of Cell Line 293T-CD40L-sBAFF

1) Firstly, 1× poly-1-lysine solution was used for treating 10 cm of a cell culture dish; after cell density reached 40% of 0 virus, a plasmid was packaged; after transfecting for 36 h, concentrated virus solution was prepared, the supernatant was filtered by a filter head (pore diameter 0.45 μm) and added to the concentrated solution for staying overnight at 4° C.; after 24 h, the supernatant of the obtained virus solution was centrifuged for 5 min at a speed of 4000 g, the obtained supernatant was discarded and 1 ml of DMEM medium was used for re-suspending;
2) the 1× poly-1-lysine solution was used for pre-treating a 12-pore plate; after the infected cell density reached about 75%, the supernatant was sucked and infection was started;
3) 1 ml of the concentrated virus solution and Polybrene with a final concentration of 6 ug/ml were added into the cells, and centrifugal infection was performed for 30 min at a speed of 1500 g; the solution was changed after 8 h of infection; and enlarged culture was performed;
4) after the cell line from enlarged culture was digested by using pancreatic enzyme, the cells were washed for two times with 1×PBS buffer solution, and positive cells in FITC channel (293T-CD40L) or double positive cells in FITC channel and PE channel (293T-CD40L-sBAFF) were sorted by using a flow cytometer; the cells were collected by a collecting tube with 2 ml DMEM; After collecting the cells, the enlarged culture was performed and the sorting was performed again; and
5) after sorting for 3-5 times, the cell positive rate was kept higher than 90% (293T-CD40L) or 80% (293T-CD40L-sBAFF), and the sorting was stopped after a positive cell proportion was kept stable for a long time.

### Separation of Human Peripheral Blood Mononuclear Cell (PBMC)

Human peripheral blood was taken from the Guangzhou blood center. The peripheral blood component was added into 1×PBS buffer solution (containing 2% of BSA and 0.5% of EDTA) and uniformly mixed, and a lymphocyte separating medium was slowly added from above to make its proportion to the buffer solution 1:1. The obtained mixture was centrifuged at a speed of 1500 rpm for 40 min. The peripheral blood mononuclear cell layer (white and flocculent, namely the albuginea layer) was carefully sucked, and placed in another centrifuge tube, 5 times of volume of the 1×PBS (containing 05% of the BSA and 2% of EDTA) was added for diluting, uniformly mixed, centrifuged at a speed of 380 g for 8 min. The above steps were repeated one time.

### Separation of B Cells Using Negative Magnetic Beads

According to a mode of adding 100 μl 1×PBS buffer solution (containing 0.5% of the BSA and 2% of EDTA) and 50 μl of CD19⁺ B cell negative sorted primary antibodies per 10⁷ PBMC cells, incubation was performed for 15 min at room temperature. 10 times of volume of the 1×PBS buffer solution was added for diluting, uniformly mixed, and centrifuged at a speed of 380 g for 8 min to discard the supernatant. 50 μL secondary antibodies with magnetic beads were added to per 10⁷ cells. After incubating at room temperature for 30 min, the mixture was sorted by the magnetic column and then centrifuged at a speed of 380 g for 8 min to obtain B cells, in which the IMDM medium containing 10% of fetal calf serum and 1% of double antibodies were added to re-suspend the cells. The total number of the cells was counted by using a counting plate. A similar negative magnetic separation was applicable to other cells.

### Separation of B Cells Using Positive Magnetic Beads

According to a mode of adding 100p of the 1×PBS buffer solution (containing 0.5% of the BSA and 2% of EDTA) and 50 μl of CD19⁺ B cell positive sorted magnetic beads per 10⁷ of the PBMC cells, the incubation was performed for 20 min at room temperature. 10 times of volume of the 1×PBS buffer solution was added for diluting, uniformly mixed, and centrifuged at a speed of 380 g for 8 min to discard the supernatant. The cell sorting was performed by passing through the magnetic column after re-suspending, the B cells was obtained after centrifugation at a speed of 380 g for 8 min, in which the RPMI1640 medium containing 10% of the fetal calf serum and 1% of the double antibodies for re-suspending. The total number of the cells was counted by using the counting plate. A similar positive magnetic separation was applicable to other cells.

### B Cell Proliferation

Firstly feeding cells were prepared. After the 12-pore plate was pre-treated with 1× poly-1-lysine solution, the cell lines 293T, 293T-CD40L-sBAFF and 293T-CD40L were laid in the 12-pore plate. While cell density was up to about 30%, the cells were stopped from growing by X-ray at an irradiation dose of 95 Gy or mitomycin C for 3 h. The cells were observed for 12 h to confirm that they were stopped from growing.

Then B cells were laid in the 12-pore plate and co-cultured with the feeding cells. After a series of experiments to explore the optimal conditions, the inventor finally determined the density of B cells to be 5×10⁵ cells/pore. B cells were suspended in the IMDM medium at a concentration of 5×10⁵/ml, in which CpG-ODN2006/2219, CsA (Cyclosporine A), IL-2, IL-10 and IL-4 at a final concentration 2 μg/mL, 0.625 μg/mL, 10 ng/mL, 10 ng/ml and 20 ng/mL, respectively were added into the culture medium. After that, cell suspension was added to the 12-pore plate at an amount of 1 mL per pore and co-cultured with the feeding cells. Every 4 days, B cells were separated from the feeding cells by using a differential centrifugation or density gradient centrifugation method, counted by trypan blue staining, and transferred to the 12-pore plate laid with new feeding cells at the density of 5×10⁵ cells/pore.

### ELISPOT Experiment

The human IFN-γ precoated ELISPOT kit(Dakewe Biotech Co., Ltd) was used in the experiment for measuring cell IFN-γ. The cell suspension at a proper concentration was added to an experiment pore. Wherein CD8⁺ T cells co-cultured with CD40L-B cells or CD40L-sBAFF-B cells were added into the experimental group, the CD8+ T cells stimulated by PHA were added into the positive control group, and the non-stimulated CD8⁺ T cells were added into the negative control group. The density of CD8⁺ T cells was 105/pore. The equal volume of RMPI 1640 was added into the background negative control group. After samples were added, the incubation was performed for 16-20 h at 37° C.

The next day, the culture medium in the pore was poured out, 4° C. of deionized water was added to lyse the cells for 10 min at 4° C. After washing the cells with 1×ELISPOT eluant for 5-7 times, antibody working solution was added and then incubated for 1 h at 37° C. Ten, the antibody was poured out, 1×ELISPOT eluant was used for washing for 5-7 times, HRP working solution was added and the incubation was performed for 1 h at 37° C.

After the end of the incubation, 1×ELISPOT eluant was used again to wash for 5-7 times, prepared AEC color developing solution was added to react for 25 min at room temperature in dark place. After the end of the reaction, the deionized water was used for washing for 5-7 times, the plate was placed in a shade place. After the plate was aired, the number of positive spots was detected by using a CTL Immunospot S5 core analyzer, and spot counting and statistical analysis were performed by CTL Immunospot software.

### Experimental Result

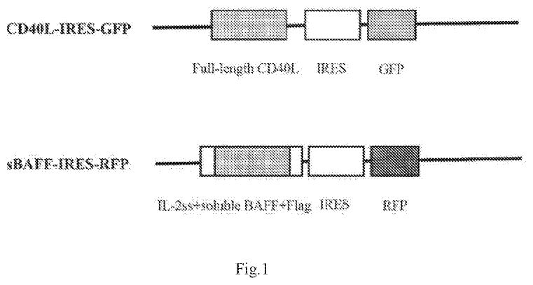

The inventor used 293T cells to construct a feeding cell line. Human 293T cells were infected by packaged slow viruses with membrane CD40L type sequence, and the stable cell line (cell line 293T-CD40L) was obtained after multiple times of screening by the flow cytometry. The amino acids 1-46 of human BAFF are intracellular domains, amino acids 47-73 are transmembrane domains, and amino acids 74-285 are extracellular domains. After BAFF was cut by fhrin protease in vivo, the amino acids 133-285 would form the soluble BAFF which is a main form of the BAFF for exerting functions. So, the inventor cloned the amino acids 133-285 of BAFF. In order to enable BAFF to be secreted out of the cells better, the inventor inserted a section of IL-2 secretion peptide at the N-terminal of BAFF by a meticulous design. At the same time, in order to conveniently detect the quantity of BAFF secreted into the supernatant later, a Flag tag sequence was fusion expressed at the C-terminal of the soluble BAFF by the inventor. The fusion protein was cloned to a slow virus vector cPPT-IRES-RFP, and then was co-transfected into the cell line 293T-CD40L with packaging plasmid psPAX and envelope protein VSVG to package the viruses. A stable GFP-RFP double positive cell line (293T-CD40L-sBAFF) was obtained after multiple times of screening RFP positive cells by the flow cytometry. The plasmid constructed in this experiment was shown in FIG. 1.

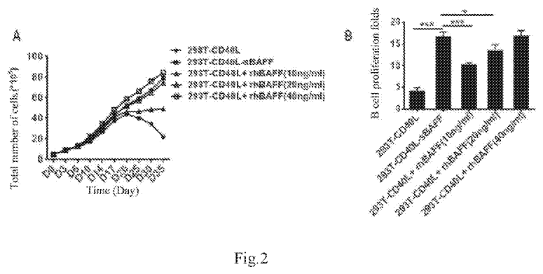

After that, the inventor used the newly constructed cell line 293T-CD40L-sBAFF to culture human peripheral blood primary B cells in vitro. The culture method was as mentioned above. In order to evaluate the proliferation effect of the cell line 293T-CD40L-sBAFF to human peripheral blood primary B cells, the inventor compared the effect on the B cells proliferation of the cell line 293T-CD40L-sBAFF with that of 293T-CD40L previously reported in literature, and that of GMP-level recombinant human soluble BAFF (rhBAFF) with final concentration of 10 ng/mL, 20 ng/mL and 40 ng/mL. The result showed that the proliferation ability on B cells proliferation of the cell line 293T-CD40L-sBAFF is superior to that of the previously reported cell line 293T-CD40L, and that of the cell line 293T-CD40L supplemented with rhBAFF with a final concentration of 10 ng/mL, as shown in FIG. 2.

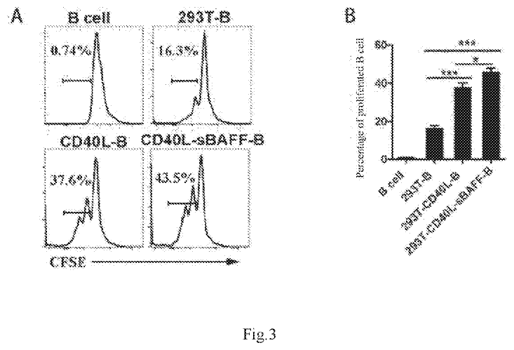

Besides the proliferation ability of B cells observed through cell counting, the inventor further analyzed a proliferation of B cells through a CFSE staining experiment. CFSE results showed that 293T-CD40L-sBAFF improved the proliferation ability of B cells, and raised the percentage of B cells with proliferation activity, indicating that the cell line 293T-CD40L-sBAFF had a stronger ability of proliferating B cells compared to the cell line 293T-CD40L, as shown in FIG. 3.

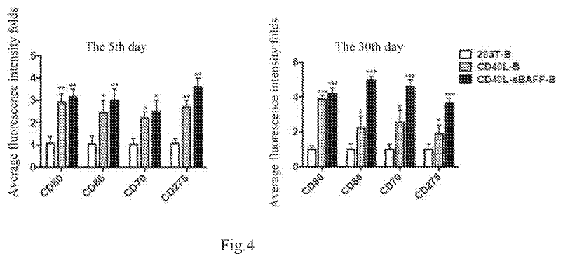

Because the activated B cells can express MHC-class molecules (MHC-I,MHC-II), co-stimulators (CD80, CD86) and adhesion molecules (CD54) have the ability to present antigens and activate T cells, and the expression level of the co-stimulators CD80 and CD86 is closely related to the antigen presenting function of B cells, after co-culture, B cells are collected and the CD80 and CD86 levels on the surface are detected by using the flow cytometry. It was indicated that after co-culture, B cells co-cultured with the cell line 293T-CD40L-sBAFF expressed the CD80 and CD86 molecules on the surface with a higher level. Besides CD80 and CD86, other co-stimulators participating in the B cell antigen presenting function, such as CD275 (ICOSLG) and CD70 (CD27L), were up-regulated in expression, as shown in FIG. 4.

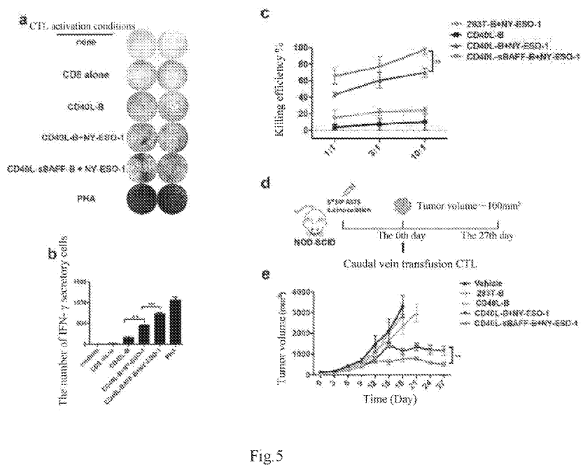

In the in vitro experiment, the inventor found that the co-stimulators participating in the cell antigen presentation were improved to some extent, and the inventor further verified whether B cells cultured by the cell line 293T-CD40L-sBAFF may improve the ability of tumor specific antigen presentation and play a role in tumor adoptive reinfusion therapy. Melanoma was an optimal tumor therapy model, and the inventor selected a melanoma specific antigen epitope NY-ESO-1, and used B cells cultured by the cell line 293T-CD40L-sBAFF to co-incubate with an antigen peptide to perform the antigen presentation. As shown in FIGS. 5a, 5b and 5c, CD8 cells represented antigen by B cells cultured by the cell line 293T-CD40L-sBAFFcould secret more IFN-γ in the ELISPOT experiment, and its killing efficiency reached 100% nearly in a cell killing experiment. In order to further verify that CD8 cells represented antigen by B cells cultured by the cell line 293T-CD40L-sBAFF also has a better anti-tumor activity in vivo, the inventor constructed an in vivo experiment model of the melanoma. As shown in FIGS. 5d and 5e, the inventor adoptive re-transferred the cultivated CTL cells into a mouse body, and measured the tumor volume. The experimental result indicated that because the ability of B cells cultured by the cell line 293T-CD40L-sBAFF, for presenting the antigen was enhanced, the cultivated CTL also had a better anti-tumor activity in vivo.

### REFERENCES

Do, R. K., Hatada, E., Lee, H., Tourigny, M. R., Hilbert, D., & Chen-Kiang, S. (2000). Attenuation of apoptosis underlies B lymphocyte stimulator enhancement of humoral immune response. The Journal of experimental medicine, 192(7), 953-964.
Garrone, P., Neidhardt, E.-M., Garcia, E., Galibert, L., Van Kooten, C., & Banchereau, J. (1995). Fas ligation induces apoptosis of CD40-activated human B lymphocytes. The Journal of experimental medicine, 182(5), 1265-1273.
Mackay, F., & Schneider, P. (2009). Cracking the BAFF code. Nat Rev Immunol, 9(7), 491-502. doi: 10.1038/nri2572
Moore, P. A., Belvedere, O., Orr, A., Pieri, K., LaFleur, D. W., Feng, P., . . . Parmelee, D. (1999). BLyS: member of the tumor necrosis factor family and B lymphocyte stimulator. Science, 285(5425), 260-263.
Schultze, J. L., Michalak, S., Seamon, M. J., Dranoff, G, Jung, K., Daley, J., . . . Nadler, L. M. (1997). CD40-activated human B cells: an alternative source of highly efficient antigen presenting cells to generate autologous antigen-specific T cells for adoptive immunotherapy. Journal of Clinical Investigation, 100(11), 2757.
Yang, M., Hase, H., Legarda-Addison, D., Varughese, L., Seed, B., & Ting, A. T. (2005). B cell maturation antigen, the receptor for a proliferation-inducing ligand and B cell-activating factor of the TNF family, induces antigen presentation in B cells. The Journal of Immunology, 175(5), 2814-2824.
Zhang, X., Li, L., Choe, J., Krajewski, S., Reed, J. C., Thompson, C., & Choi, Y. S. (1996). Up-regulation of Bcl-x L expression protects CD40-activated human B cells from Fas-mediated apoptosis. Cellular immunology, 173(1), 149-154.

1. A secretory BAFF peptide, comprising an amino acid sequence consisting of amino acids 133-285 of BAFF and an IL-2 peptide at N-terminal thereof, wherein a sequence of the amino acids 133-285 of BAFF is
AVQGPEETVTQDCLQLIADSETPTIQKGSYTFVPWLLSFKRGSALEEKEN
KILVKETGYFFIYGQVLYTDKTYAMGHLIQRKKVHVFGDELSLVTLFRCI
QNMPETLPNNSCYSAGIAKLEEGDELQLAIPRENAQISLDGDVTFFGALK
LL,and
an amino acid sequence of the IL-2 peptide is
MYRMQLLSCIALSLALVTNS.

2. An expression vector for a secretory BAFF peptide, comprising a nucleotide sequence expressing the secretory BAFF peptide according to claim 1.

3. The expression vector according to claim 2, further comprising a nucleotide sequence expressing a membrane type CD40L.

4. A cell for expressing a secretory BAFF peptide, wherein the cell is transfected with the expression vector according to claim 2.

5. The cell according to claim 4, wherein the cell is further transfected with a vector expressing a membrane type CD40L.

6. The cell according to claim 4, wherein the cell is transfected with the expression vector according to claim 3.

7. The cell according to claim 4, wherein the cell is 293T cell.

8. A method for proliferating human peripheral blood B cells in vitro, comprising a step of co-culturing human peripheral blood B cells with cells simultaneously expressing a secretory BAFF peptide and a membrane type CD40L.

9. The method for proliferating human peripheral blood B cells in vitro according to claim 8, wherein the human peripheral blood B cells are cultured in insulin-transferrin-supporting IMDM medium supplemented with CpG2006/2219 and ciclosporin A for stimulating B cell proliferation, and with human IL-4, IL-2 and IL-10 for maintaining cell growth.
